(19) United States
(12) Patent Application Publication
(10) Pub. No.: US 2014/0121490 A1
(43) Pub. Date: May 1, 2014

(54) ACOUSTO-OPTIC IMAGING DEVICE

(71) Applicant: PANASONIC CORPORATION, Osaka (JP)

(72) Inventors: Masahiko HASHIMOTO, Osaka (JP); Ushio SANGAWA, Nara (JP); Yuriko KANEKO, Nara (JP); Takuya IWAMOTO, Osaka (JP); Takahiro KAMAl, Kyoto (JP)

(73) Assignee: PANASONIC CORPORATION, Osaka (JP)

(21) Appl. No.: 14/147,096

(22) Filed: Jan. 3, 2014

Related U.S. Application Data
(63) Continuation of application No. PCT/JP2013/003304, filed on May 24, 2013.

(30) Foreign Application Priority Data
Jun. 4, 2012 (JP) 2012-126998

(51) Int. Cl. A61B 5/00

(52) U.S. Cl. CPC A61B 5/0097
USPC 600/407

(57) ABSTRACT

An acousto-optic imaging device includes: an ultrasonic wave transmitter for transmitting a divergent ultrasonic wave into a subject; an acoustic lens for converging a reflection ultrasonic wave derived from the ultrasonic wave from the subject; an acousto-optic cell including an acousto-optic propagation medium section which has a smaller sound velocity than the subject and through which the reflection ultrasonic wave converged by the acoustic lens propagates; a light source for emitting convergent light so as to irradiate the reflection ultrasonic wave propagating through the acousto-optic propagation medium section in a direction not parallel to a traveling direction of the reflection ultrasonic wave; and an image formation optical system for detecting Bragg diffracted light of the convergent light which is produced in the acousto-optic propagation medium section and converting the detected Bragg diffracted light to an electric signal.

This is a continuation of International Application No. PCT/JP2013/003304, with an international filing date of May 24, 2013, which claims priority of Japanese Patent Application No. 2012-126998, filed on Jun. 4, 2012, the contents of which are hereby incorporated by reference.

### BACKGROUND

1. Technical Field

The present invention relates to an acousto-optic imaging device and particularly to an acousto-optic imaging device for obtaining an ultrasonic echo from an object as an optical image.

2. Description of the Related Art

Ultrasonic diagnostic apparatuses are capable of noninvasively obtaining an image of an inner portion of the body of a patient or subject. Therefore, conventionally, the ultrasonic diagnostic apparatuses have been widely used in the medical fields. The ultrasonic diagnostic apparatus irradiates an inner portion of a subject with an ultrasonic wave and detects a reflected ultrasonic echo, thereby obtaining a two-dimensional or three-dimensional image of a tissue or organ inside the subject. Such an ultrasonic diagnostic apparatus generally employs, for transmission and reception of ultrasonic waves, a probe in which a plurality of piezoelectric elements are one- or two-dimensionally arranged (transducer array probe) as disclosed in Japanese Patent No. 54-34580 (hereinafter, referred to as “Patent Document 1”). Signal processing called “beam forming”, such as retardation processing of a driving signal for driving the plurality of piezoelectric elements, is carried out such that the inner portion of the body of the subject is scanned with ultrasonic waves transmitted from the plurality of piezoelectric elements as an ultrasonic beam. Likewise, signal processing is carried out such that ultrasonic echoes received by the plurality of piezoelectric elements are detected as an ultrasonic beam corresponding to the scanning. Since information inside the body is obtained by scanning with an ultrasonic beam under the control of an electric circuit, such an ultrasonic diagnostic apparatus is called “electronic scanning ultrasonic diagnostic apparatus”.

### SUMMARY

Highly-developed medical technology has created demand for three-dimensional imaging of a tissue or organ inside a subject with higher resolution. To this end, it is necessary to further increase the number of piezoelectric elements of the probe. However, when the number of the piezoelectric elements is increased, a greater throughput for signal processing is necessary for the beam forming, so that it is difficult to obtain an image in real time, or a signal processing circuit which has a very high computational power is necessary, so that the size of the apparatus increases or the cost of the apparatus increases.

One of the objects of a nonlimiting exemplary acousto-optic imaging device of the present invention is to provide an acousto-optic imaging device which is capable of imaging a large area inside an organism's body without using a signal processing circuit which has a high computational power.

An acousto-optic imaging device disclosed in the present application includes: an ultrasonic wave transmitter for transmitting a divergent ultrasonic wave into a subject; an acoustic lens for converging a reflection ultrasonic wave derived from the ultrasonic wave from the subject; an acousto-optic cell including an acousto-optic propagation medium section which has a smaller sound velocity than the subject and through which the reflection ultrasonic wave converged by the acoustic lens propagates; a light source for emitting convergent light so as to irradiate the reflection ultrasonic wave propagating through the acousto-optic propagation medium section in a direction not parallel to a traveling direction of the reflection ultrasonic wave; and an image formation optical system for detecting Bragg diffracted light of the convergent light which is produced in the acousto-optic propagation medium section and converting the detected Bragg diffracted light to an electric signal.

According to an acousto-optic imaging device disclosed in the present application, convergent light is diffracted by a reflected ultrasonic wave obtained from the inner portion of a subject, whereby the inner portion of the subject can be optically imaged.

These general and specific aspects may be implemented using a system, a method, and a computer program, and any combination of systems, methods, and computer programs.

Additional benefits and advantages of the disclosed embodiments will be apparent from the specification and Figures. The benefits and/or advantages may be individually provided by the various embodiments and features of the specification and drawings disclosure, and need not all be provided in order to obtain one or more of the same.

### BRIEF DESCRIPTION OF THE DRAWINGS

FIG. 1 is a schematic configuration diagram showing the first embodiment of the acousto-optic imaging device of the present invention.

FIGS. 2A, 2B, 2C, 2D 2E and 2F are diagrams for illustrating the operation of the acousto-optic imaging device shown in FIG. 1, showing the transition of an ultrasonic wave traveling through a subject and an acousto-optic imaging device with the passage of time.

FIG. 3 is a diagram showing the positional relationship between an acoustic image formed in an acousto-optic cell of the acousto-optic imaging device shown in FIG. 1 and convergent light used for diffraction.

FIG. 4 is a diagram showing an experimental result carried out for confirmation of the operation of the first embodiment.

FIG. 5 is a diagram showing an experimental result carried out for confirmation of the operation of the first embodiment.

FIG. 6 is a schematic configuration diagram showing the second embodiment of the acousto-optic imaging device of the present invention.

FIGS. 7A, 7B, 7C and 7D are diagrams showing an experimental result carried out for confirmation of the operation of the second embodiment.

FIGS. 8A, 8B, 8C and 8D are diagrams showing an experimental result carried out for confirmation of the operation of the second embodiment.

FIG. 9 is a diagram for illustrating the principle of Bragg diffraction which is disclosed in Non-patent Document 1.

FIG. 10 is a diagram for schematically illustrating an acousto-optic effect which is attributed to Bragg diffraction.

### DETAILED DESCRIPTION

The inventors of the present application studied the method for two- or three-dimensionally obtaining an image rather than scanning an internal tissue of a subject with an ultrasonic wave as in conventional ultrasonic diagnostic apparatuses. As a result, the inventors arrived at utilizing an acousto-optic effect, which is interaction between an ultrasonic wave and light, for obtaining an image of an internal tissue of the subject.

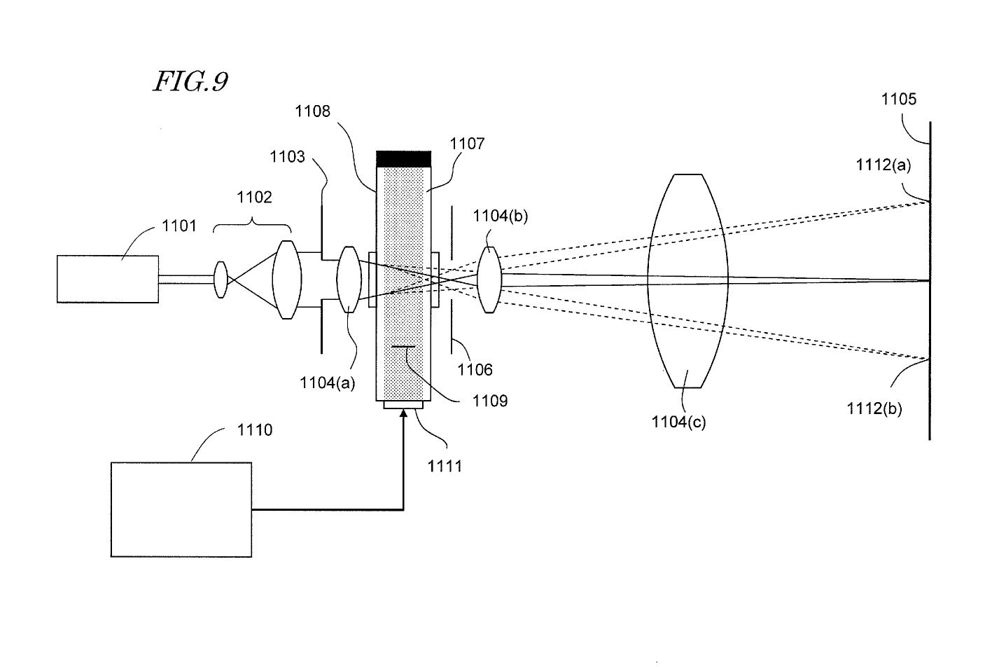

Specifically, as disclosed in Non-patent Document 1, A. Korpel, “Visualization of the cross section of a sound beam by Bragg diffraction of light,” Applied Physics Letters, vol. 9. no. 12, pp. 425-427, 15 Dec. 1966, it was found that utilizing Bragg diffraction which is attributed to uneven density caused in a transmission medium section by an ultrasonic wave enables imaging of an internal tissue of the subject. FIG. 9 shows a configuration disclosed in Non-patent Document 1. As shown in FIG. 9, a monochromatic light beam emitted from a laser light source 1101 is converted by a beam expander 1102 and an aperture 1103 into a large-diameter plane wave light beam. The plane wave light beam passes through an acousto-optic cell 1108 and cylindrical lenses 1104(a), 1104(b), and 1104(c), and is projected onto a screen 1105. The optical system that is formed by the cylindrical lenses 1104(a), 1104(b), and 1104(c) has an asymmetrical configuration in which the converged state is different between a direction horizontal to the drawing sheet and a direction vertical to the drawing sheet. Therefore, this optical system has an astigmatism.

The focal length of the cylindrical lens 1104(a) is determined such that the plane wave light beam outgoing from the beam expander 1102 is focused at the position of a focal plane 1106 on a plane which is parallel to the drawing sheet of FIG. 9 as represented by solid lines in FIG. 9. The light beam which has passed through the focal plane 1106 then diverges. The diverging light beam is converged by the cylindrical lens 1104(b) so as to be focused again on the screen 1105.

On the other hand, in a plane which includes the optical axis of the beam expander 1102 and which is perpendicular to the drawing sheet of FIG. 9, the plane wave light beam which has passed through the beam expander 1102 remains as a collimated light beam when it is incident on the cylindrical lens 1104(c). Thereafter, due to the light-condensing function of the cylindrical lens 1104(c), the light beam is focused on the screen 1105. The positions and lens planes of the cylindrical lenses 1104(a), 1104(b), and 1104(c) are determined such that the optical system formed by these lenses has equal image magnification rates in the direction parallel to the drawing sheet of FIG. 9 and in the direction perpendicular to the drawing sheet of FIG. 9 (the magnification rate=the size of a imaging object 1109/the size of an image on the screen 1105).

The imaging object 1109 is immersed in the acousto-optic cell 1108 which is filled with water 1107. The imaging object 1109 is irradiated with a monochromatic ultrasonic plane wave which is supplied through the water 1107 from an ultrasonic transducer 1111 that is driven by a signal source 1110. In this process, an ultrasonic scattered wave is produced at the imaging object 1109, and the scattered wave propagates through a region of the water 1107 through which the monochromatic light from the laser light source 1101 passes. The major guided mode of the ultrasonic wave propagating in the water is a compression wave (longitudinal wave), and therefore, a refractive index distribution which is identical with the acoustic pressure distribution in the water 1107, i.e., identical with the ultrasonic wavefront, is caused in the water.

Since the ultrasonic scattered wave is monochromatic (=an ultrasonic wave which has a single frequency), a refractive index distribution caused in the water 1107 at a certain moment is a one-dimensional grating in the shape of a sine wave which is repeated at an ultrasonic wavelength. Thus, due to the one-dimensional grating, diffracted light is produced (of which only the ±1st order diffracted light beams are shown in the drawing). The diffracted light appears as a light spot on the screen 1105. The luminance of the light spot is proportional to the variation of the refractive index of the one-dimensional grating, i.e., the ultrasonic acoustic pressure.

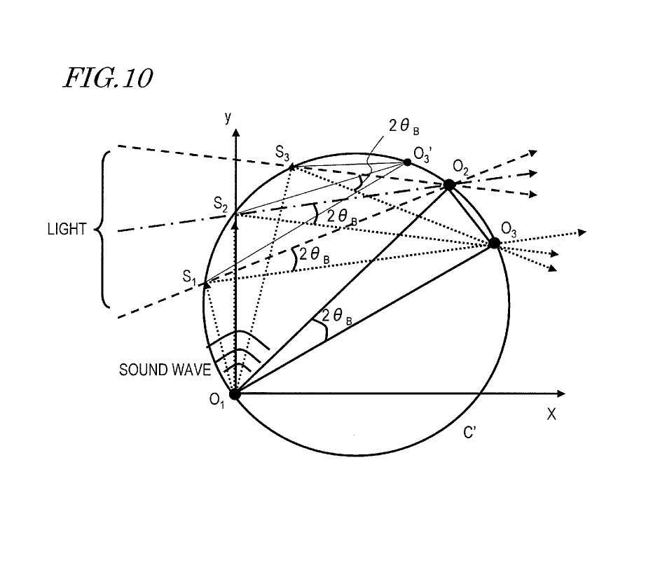

FIG. 10 schematically shows an acousto-optic effect which is attributed to Bragg diffraction. In FIG. 10, point O₁ is a point sound source (Huygens sound source), which radiates a spherical wave.

Light converges at point O₂ with line segment S₁O₂ and line segment S₃O₂ in FIG. 10 being the edges of the light beam. Due to uneven density caused in an acoustic medium by an ultrasonic wave, the −1st order Bragg diffracted light is produced at each point on arc S₁S₃ in a direction which satisfies Bragg angle θ_{B} and converges at point O₃. By a geometric optics analysis, it is deduced that points O₁, O₂, and O₃ are on the same circumference C′. Triangle O₁O₂O₃ is an isosceles triangle. Angle O₂O₁O₃ is 2θ_{B}.

In this way, convergent light is allowed to affect the diffraction grating which is produced by an acoustic wave that is a spherical wave, and a spherical light source (arc S₁S₃) is virtually produced by utilizing the angle dependence of Bragg diffraction, so that, at point O₃, point sound source O₁ can be produced as an optical image. The ratio between side O₁O₂ and side O₂O₃ of triangle O₁O₂O₃ represents the reduction rate in an image produced by Bragg diffracted light, where the reduction rate is O₂O₃/O₁O₂=λ/Λ. Here, Λ is the wavelength of the sound wave, and λ is the wavelength of the light. These principles also apply to the +1st order diffraction image. In FIG. 10, an optical image of point sound source O₁ is produced at point O₃′.

Image formation which is based on such a principle is realized by an optical image formation effect of the converging optical system as in conventional optical cameras, as appreciated from the above description which has been provided with reference to FIG. 10. That is, an image of an internal tissue of a subject can be formed without using a group of receivers, a probe including a large number of ultrasonic transducers which have equal wave transmission/reception characteristics, and a high-speed, large-scale arithmetic circuit for signal processing that is to be carried out on a group of received signals output from the group of receivers, such as beam forming, which are necessary in conventional electronic scanning ultrasonic diagnostic apparatuses.

However, Non-patent Document 1 only discloses an acousto-optic effect which is attributed to Bragg diffraction and provides no suggestion about how to realize imaging of an internal tissue of an organism's body by the utilization of the acousto-optic effect. The inventors of the present application examined details of the techniques disclosed in Non-patent Document 1. According to the techniques of Non-patent Document 1, the frequency of an ultrasonic wave used is high, specifically, not less than 15 MHz. This is because the acousto-optic cell is formed by an aqueous medium, and the relationship between the sound velocity in water (about 1500 m/s) and the wavelength of an ultrasonic wave restricts the conditions under which Bragg diffraction occurs. In the organism's body, the absorption attenuation increases substantially in proportion to the frequency, and therefore, in order to form an image of a deep inner portion of a subject, using an ultrasonic wave at a frequency of 10 MHz or lower is preferred. Thus, when the configuration disclosed in Non-patent Document 1 is used without modification, it is difficult to obtain an image of an internal tissue of the body of a subject.

Further, Non-patent Document 1 utilizes a scattered wave which is produced near the contour of the imaging object 1109. However, in the case of such a method, forming a high-definition image of an internal tissue of the organism's body is difficult.

Further, according to the techniques disclosed in Non-patent Document 1, the range from which an image can be obtained is very narrow, and the distance from a region of interaction with light is restricted. For this, as shown in FIG. 10, according to such an image formation condition that the sound source denoted by point O₁, point O₂ that is the position of convergence of light, and point O₃ that is the position of convergence of diffracted light are on the same circumference, when the distance between light converging at point O₂ and point O₁ that is the sound source (the Z direction in FIG. 10) increases, the arc becomes larger in proportion to that distance. Therefore, conversion of an ultrasonic wave from a deep inner portion of an organism's body into an image requires a large size acousto-optic cell. This is also a problem in the case where point O₃ that is the sound source moves in a horizontal direction (X direction in FIG. 10). As a result, it is difficult to obtain an image over a wide range inside the organism's body.

The inventors of the present application examined the above problems in detail and conceived a novel acousto-optic imaging device. The summary of an embodiment of the acousto-optic imaging device of the present invention is as described below.

An acousto-optic imaging device disclosed in the present application includes: an ultrasonic wave transmitter for transmitting a divergent ultrasonic wave into a subject; an acoustic lens for converging a reflection ultrasonic wave derived from the ultrasonic wave from the subject; an acousto-optic cell including an acousto-optic propagation medium section which has a smaller sound velocity than the subject and through which the reflection ultrasonic wave converged by the acoustic lens propagates; a light source for emitting convergent light so as to irradiate the reflection ultrasonic wave propagating through the acousto-optic propagation medium section in a direction not parallel to a traveling direction of the reflection ultrasonic wave; and an image formation optical system for detecting Bragg diffracted light of the convergent light which is produced in the acousto-optic propagation medium section and converting the detected Bragg diffracted light to an electric signal.

The convergent light is emitted so as to irradiate a portion of the acousto-optic propagation medium section through which the reflection ultrasonic wave in a state of a divergent wave after convergence is propagating.

The convergent light is emitted so as to irradiate a portion of the acousto-optic propagation medium section through which the reflection ultrasonic wave in a state of a convergent wave is propagating.

The acousto-optic propagation medium section includes an inert perfluorocarbon fluid.

The acousto-optic propagation medium section includes an inert hydrofluoroether fluid.

The acousto-optic propagation medium section includes a silica nanoporous element.

The acousto-optic imaging device further includes a wave reception standoff for supporting the acoustic lens, wherein a convergence point on a subject side of the acoustic lens occurs inside the wave reception standoff.

The acousto-optic imaging device further includes a wave transmission standoff for supporting the ultrasonic wave transmitter, wherein the ultrasonic wave transmitter transmits a converging ultrasonic wave, and a point of the convergence occurs inside the wave transmission standoff.

Hereinafter, embodiments of the acousto-optic imaging device of the present invention will be described in detail.

### First Embodiment

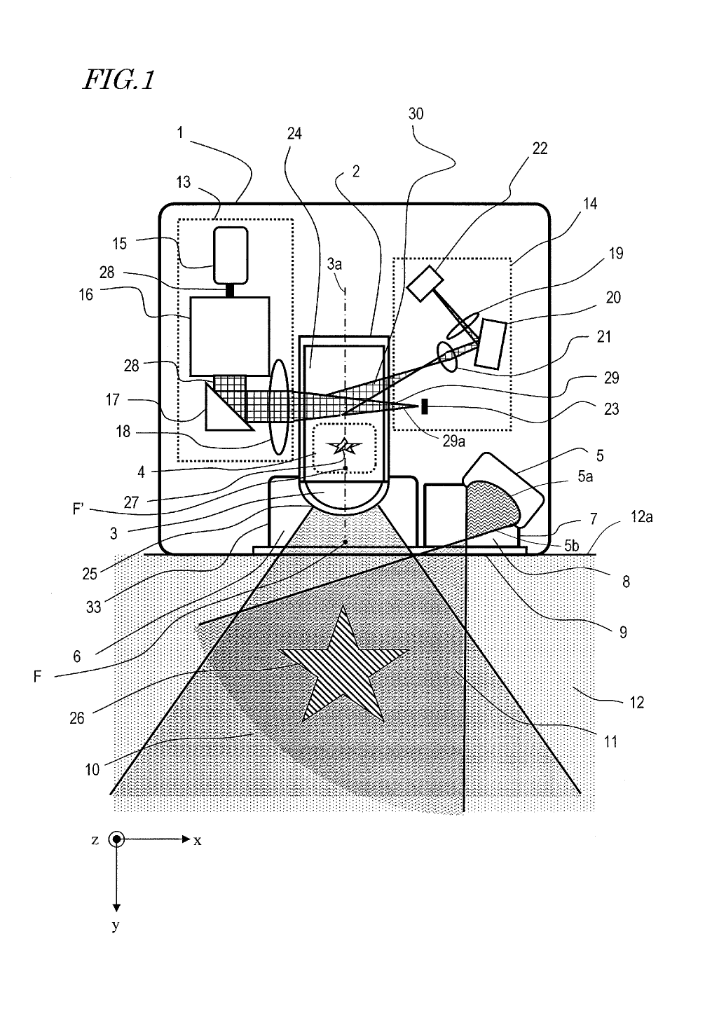

FIG. 1 is a schematic diagram showing the first embodiment of the acousto-optic imaging device of the present invention. The acousto-optic imaging device 1 shown in FIG. 1 produces an image of an internal tissue of a subject 12, such as a human being or animal, for example. For the sake of simple illustration, an organ inside the subject 12 is schematically shown as a star-shaped reflector 26. In the drawings mentioned below, the reflector 26 is shown as a two-dimensional object which is parallel to the drawing sheet but, however, the reflector 26 is, in general, a three-dimensional object. When observing an actual human being or animal, an ultrasonic wave is reflected at a portion in which there is a difference in acoustic impedance, such as an organ or tissue inside the body, as in the conventional ultrasonic diagnostic apparatuses. Therefore, each tissue inside the subject can be converted into an image as the reflector 26 as in the conventional ultrasonic diagnostic apparatus.

The acousto-optic imaging device 1 includes an ultrasonic wave transmitter 5, an acoustic lens 3, an acousto-optic cell 2, a light source 13, and an image formation optical system 14.

The acousto-optic imaging device 1 transmits an ultrasonic wave from the ultrasonic wave transmitter 5 to the subject 12 and receives a reflection ultrasonic wave reflected by the subject 12 at the acoustic lens 3. The acoustic lens 3 converges the received reflection ultrasonic wave. The converged reflection ultrasonic wave propagates through the acousto-optic cell 2. Convergent light is emitted from the light source 13 to irradiate the reflection ultrasonic wave propagating through the acousto-optic cell 2. As a result, diffraction by the reflection ultrasonic wave is caused. The image formation optical system 14 detects produced diffracted light and converts it to an electric signal, thereby obtaining an image of an inner portion of the subject 12. The respective components will be described in detail below.

<Ultrasonic Wave Transmitter 5>

The ultrasonic wave transmitter 5 transmits an ultrasonic wave to the subject 12. The ultrasonic wave transmitted by the ultrasonic wave transmitter 5 is preferably a divergent wave which diverges in the subject. Since an image of an internal tissue of the subject within the range 11 of the transmitted ultrasonic wave can be formed, larger part of the inner portion of the subject can be imaged as the range of divergence of the transmitted ultrasonic wave increases.

In the present embodiment, the ultrasonic wave transmitter 5 transmits a convergent ultrasonic wave from a transmission plane 5a and transmits an ultrasonic wave, which diverges from a convergence point 5b that is distant from the transmission plane 5a by a predetermined distance, to the inner portion of the subject 12. To this end, the acousto-optic imaging device 1 further includes a standoff 7 for wave transmission. The wave transmission standoff 7 supports the ultrasonic wave transmitter 5 such that the convergence point 5b occurs inside the wave transmission standoff 7. This arrangement prevents the convergence point 5b, at which the ultrasonic wave converges to cause a high energy density, from occurring inside the subject. Further, the ultrasonic wave which is in a divergent state at a surface 12a of the subject 12 can be employed to irradiate the subject 12, and therefore, an image can be obtained from a wide area even in an inner portion of the subject 12 near the surface 12a. When the ultrasonic wave transmitter 5 is supported such that the transmitted ultrasonic wave propagates through the wave transmission standoff 7, the wave transmission standoff 7 is filled with a coupling medium 8, such as degassed water or various oils, such that the transmitted ultrasonic wave undergoes a small attenuation.

In the present embodiment, the ultrasonic wave transmitter 5 transmits a convergent ultrasonic wave from the transmission plane 5a but may transmit a divergent ultrasonic wave directly from the transmission plane 5a. In this case, the wave transmission standoff 7 may not be used.

The ultrasonic wave transmitted by the ultrasonic wave transmitter 5 may be, for example, a burst wave. The burst wave has such a temporal waveform that sinusoidal or rectangular waveforms which have constant amplitudes and frequencies, such as a plurality of waves which have identical sinusoidal waveforms, continue for a predetermined period of time. The burst wave preferably has such a number of waves that Bragg diffraction can occur. For example, the burst wave preferably has about 3 or 4 waves to 20 waves. The transmitted ultrasonic wave preferably has such a frequency that the attenuation is small inside the subject 12 that is an object of imaging. Specifically, it is preferably from several MHz to 15 MHz.

By repeatedly transmitting ultrasonic waves, images of an inner portion of the subject 12 can be obtained one after another. In this case, the timing of repetition can be arbitrarily set. As previously described, according to the present embodiment, in optically obtaining images of an inner portion of the subject 12, a single image can be obtained without the necessity of enormous signal processing. Thus, images can be obtained at a high speed. Therefore, the timing of repetition of ultrasonic wave transmission can be set according to the speed of displacement of an internal tissue of the subject 12 or the purpose of imaging. The timing of repetition may be, for example, from several Hz to several KHz.

The ultrasonic wave transmitted by the ultrasonic wave transmitter 5 is transmitted to the subject 12 via a window 9 which is in contact with the subject 12. To prevent reflection of the ultrasonic wave at the surface 12a of the subject 12 and allow the ultrasonic wave to efficiently enter into the subject 12, matching gel or cream, which would be applied over the surface 12a of the subject 12 or a surface of a probe in the conventional ultrasonic diagnostic apparatuses, may be provided between the window 9 and the subject 12. Alternatively, an acoustic impedance matching layer may be employed. The matching gel or cream or the acoustic impedance matching layer may be used in order to efficiently guide a reflection ultrasonic wave obtained from the subject 12 to the acoustic lens 3 via the window 9.

<Acoustic Lens 3>

The acoustic lens 3 receives and converges a reflection ultrasonic wave produced by reflection inside the subject 12 of an ultrasonic wave transmitted from the ultrasonic wave transmitter. In the present embodiment, the acoustic lens 3 is a refractive lens and has a shape of rotational symmetry about an acoustic axis 3a. Thus, according to the Snell's law, based on the shape of the acoustic lens 3, the reflection ultrasonic wave is converged three-dimensionally (in the x-, y-, and z-directions). The acoustic lens 3 has focal points F and F′ on the subject 12 side and the acousto-optic cell 2 side, for example. In the present embodiment, the sound velocity in the acoustic lens is smaller than the sound velocity in the subject 12, and therefore, a surface of the acoustic lens 3 on the subject 12 side has a convex shape toward the outward direction thereof. This arrangement enables the reflection ultrasonic wave incoming from the subject 12 side to converge. In the case where the sound velocity in the acoustic lens is larger than the sound velocity in the subject 12, the surface of the acoustic lens 3 on the subject 12 side has a concave shape toward the outward direction thereof.

As will be described in detail below, the acoustic lens 3 is preferably held relative to the subject 12 such that the convergence point (focal point) on the subject 12 side occurs outside the subject 12. With this arrangement, the reflection ultrasonic wave obtained from a region inside the subject 12 which is near the surface 12a can also be converged at a convergence point on a side of the acoustic lens 3 which is opposite to the subject 12. Therefore, the acousto-optic imaging device 1 may further include a wave reception standoff 33 for supporting the acoustic lens 3 such that the convergence point on the subject 12 side of the acoustic lens 3 occurs inside the wave reception standoff 33. This realizes the above-described positional relationship between the acoustic lens 3 and the subject 12. Note that, however, particularly when it is not necessary to obtain an image of a portion inside the subject 12 near the surface 12a, for example, when one intends to obtain an image of a deep inner portion of the subject 12, the convergence point on the subject 12 side of the acoustic lens 3 may occur inside the subject 12. The acoustic lens may be formed by an elastic element whose propagation loss for the acoustic wave is small, for example, a silica nanoporous element, water, a fluorine inactive liquid such as Fluorinert, polystyrene, etc. Further, the wave reception standoff 33 is filled with a coupling medium 6 which causes a small attenuation of the transmitted ultrasonic wave, such as degassed water or various oils.

<Acousto-Optic Cell 2>

The acousto-optic cell 2 includes an acousto-optic propagation medium section 24. The acousto-optic propagation medium section 24 has a smaller sound velocity than the subject 12 and is placed relative to the acoustic lens 3 such that the reflection ultrasonic wave converged by the acoustic lens 3 propagates therethrough. As shown in FIG. 1, the reflection ultrasonic wave propagates along the acoustic axis 3a of the acoustic lens 3, and therefore, the acousto-optic propagation medium section 24 is preferably placed at a position which includes the acoustic axis 3a.

The acousto-optic propagation medium section 24 is formed by a liquid or isotropic elastic element which causes a small propagation attenuation in the propagating reflection ultrasonic wave and which has transparency to the convergent light 29 emitted from the light source 13. The acousto-optic propagation medium section 24 is formed by, for example, a silica nanoporous element, a fluorine solvent such as Fluorinert, etc. Since the sound velocity of the acousto-optic propagation medium section 24 is smaller than the sound velocity of the subject 12, Bragg diffracted light can be produced even when the wavelength of the ultrasonic wave propagating through the acousto-optic propagation medium section 24 is shortened, and the frequency is low.

<Light Source 13>

The light source 13 emits the convergent light 29 for irradiating the reflection ultrasonic wave propagating through the acousto-optic propagation medium section 24 in a direction which is not parallel to the traveling direction of the reflection ultrasonic wave. To this end, the light source 13 includes, for example, a monochromatic light source 15, a beam expander 16, a reflection mirror 17, and a cylindrical lens 18.

The monochromatic light source 15 produces a highly coherent light beam 28. Light rays in the light beam 28 have equal wavelengths and equal phases. The monochromatic light source 15 used may be, for example, a gas laser, which is typified by a He—Ne laser, a solid-state laser, a semiconductor laser which is narrow-banded by an external resonator, or the like. The light beam emitted by the monochromatic light source 15 may be continuous or may be a pulsed light beam whose emission timing can be controlled. When the wavelength of the produced light beam may be set within such a wavelength band that the propagation loss in the acousto-optic propagation medium section 24 is small, a high luminance image can be obtained. For example, when the acousto-optic propagation medium section 24 used is a silica nanoporous element, a laser which has a wavelength of not less than 600 nm may be used. In the present embodiment, the diameter of the light beam emitted from the monochromatic light source 15 is increased by the beam expander 16. The light beam is then reflected by the reflection mirror 17 and is thereafter converted by the cylindrical lens 18 into convergent light. The cylindrical lens 18 has a lens shape so as to converge light at a plane which is parallel to the drawing sheet of FIG. 1, for example, and has a pole-like shape which is elongated along a direction perpendicular to the drawing sheet (z-direction). Thus, light which has traveled through the beam expander 16 is converged in directions parallel to the drawing sheet (x-y plane) and is not converged in the z-direction.

In the present embodiment, the convergent light 29 is emitted from the light source 13 for irradiating the reflection ultrasonic wave in a region of the acousto-optic propagation medium section 24 which is opposite to the acoustic lens 3 relative to the convergence point of the acoustic lens 3. The convergent light 29 is emitted for irradiating the acousto-optic propagation medium section 24 in the traveling direction of the reflection ultrasonic wave, i.e., in a direction which is not parallel to the acoustic axis 3a of the acoustic lens 3.

<Image Formation Optical System 14>

The image formation optical system 14 detects Bragg diffracted light of the convergent light which is produced in the acousto-optic propagation medium section 24 and converts the diffracted light to an electric signal which is then output therefrom. The image formation optical system 14 includes, for example, a cylindrical lens 21, a mirror 20, a cylindrical lens 19, and an image sensor 22. When stray light, or the like, can occur in the image formation optical system 14 due to non-diffracted convergent light 29, a blocking plate 23 may be provided for blocking the convergent light 29.

The focal length of the cylindrical lens 21 is set such that diffracted light reflected by the mirror 20 is focused on a light receiving plane of the image sensor 22 which is on a plane parallel to the drawing sheet of FIG. 1. The focal length of the cylindrical lens 19 is set such that light is focused on a light receiving plane of the image sensor 22 which is on a plane parallel to the drawing sheet of FIG. 1. When necessary, image processing is carried out on the output from the image formation optical system 14, and the resultant signal is input to a display device, at which an image of an internal tissue of the subject 12 is then displayed.

<Operation of the Acousto-Optic Imaging Device 1>

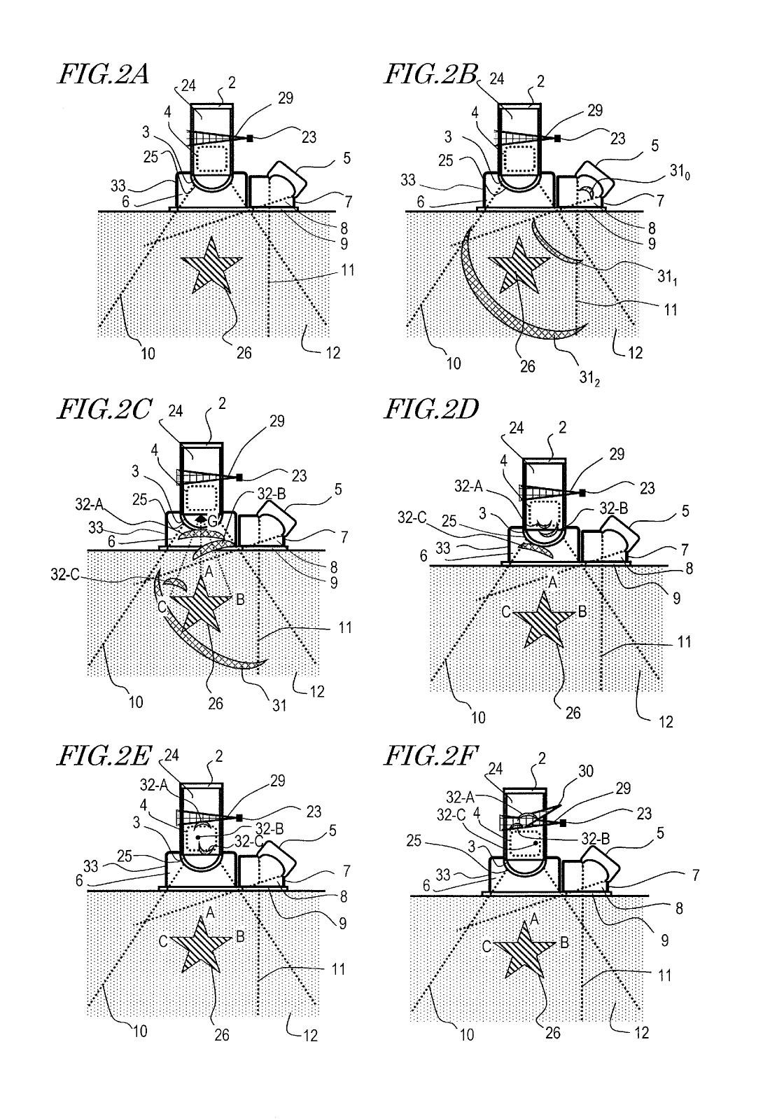

An operation of the acousto-optic imaging device 1 is described with reference to FIG. 1 and FIGS. 2A to 2F. In FIGS. 2A to 2F, for the sake of comprehensibility, components which have no direct relation to the description are not shown.

<FIG. 2A>

FIG. 2A shows the state of the acousto-optic imaging device 1 before the ultrasonic wave transmitter 5 transmits an ultrasonic wave. In the acousto-optic cell 2, the Bragg diffracted light 30 is not produced.

<FIG. 2B>

FIG. 2B shows a time variation of the ultrasonic wave 31 transmitted from the ultrasonic wave transmitter 5. The ultrasonic wave transmitted from the ultrasonic wave transmitter 5 propagates through the acoustic medium section in the order of ultrasonic waves 31₀, 31₁, 31₂ with the passage of time. For the sake of convenience of description, a reflection ultrasonic wave which is concurrently produced is omitted. The ultrasonic wave 31₀ transmitted from the ultrasonic wave transmitter 5 once converges at the convergence point 5b and then diverges. Therefore, the ultrasonic waves 31₁, 31₂ spread wider as they go deeper into the subject 12. As described above, the ultrasonic waves 31₀, 31₁, 31₂ represent the ultrasonic wave 31, which are formed by the same burst wave at different times, and it is not meant that they exist concurrently.

<FIG. 2C>

FIG. 2C shows how the ultrasonic wave 31 is reflected by the reflector 26 inside the subject 12 when it passes through the reflector 26, so that a reflection wave is produced.

When the ultrasonic wave 31 reaches the reflector 26 inside the subject 12, the ultrasonic wave 31 is reflected at respective points that form the reflector 26 so that reflection ultrasonic waves are produced. These reflection ultrasonic waves are spherical waves diverging from the respective points as the point sources. FIG. 2C illustrates that, when the ultrasonic wave 31 passes through the reflector 26, part of the wave is reflected at vertexes A, B, C, and reflection ultrasonic waves 32-A, 32-B, 32-C are produced. The ultrasonic wave 31 is also reflected at the other portions of the reflector 26 than the vertexes A, B, C, although the reflection at the other portions is not shown for the sake of comprehensibility. As described above, the reflection ultrasonic waves 32-A, 32-B, 32-C are spherical waves and propagate from the vertexes A, B, C in all of the directions, although only components traveling toward the acoustic lens 3 are shown in the drawing. The components traveling toward the acoustic lens 3 propagate along line segments extending from the vertexes A, B, C to the center of the curvature of the acoustic lens, G.

Since the vertex A is closer to the acoustic lens 3 than the vertexes B, C, the reflection ultrasonic wave 32-A reflected at the vertex A propagates to the acoustic lens 3 earlier than the others. Next, the reflection ultrasonic wave 32-B from the vertex B to which the ultrasonic wave 31 reaches earlier than the vertex C propagates to the acoustic lens 3. Lastly, the reflection ultrasonic wave 32-C from the vertex C propagates to the acoustic lens 3.

The propagation range of the reflection ultrasonic wave 32-C is smaller than those of the reflection ultrasonic waves 32-A, 32-B. This represents that the reflection ultrasonic wave 32 diverges as it propagates. The reflection ultrasonic waves 32-A, 32-B, 32-C propagate from the positions of the vertexes A, B, C of the reflector 26. The order in which the reflection ultrasonic waves 32-A, 32-B, 32-C reach the acoustic lens 3 depends on the distances between the ultrasonic wave transmitter 5 and the vertexes A, B, C of the reflector 26′ and the distances between the vertexes A, B, C and the acoustic lens 3. Since the reflection ultrasonic waves 32-A, 32-B, 32-C are spherical waves, they diverge as they propagate toward the acoustic lens 3. Therefore, the reflection ultrasonic wave 32-C that represents a wave immediately after reflection from point C is shown as a small wave.

<FIG. 20>

FIG. 2D shows a state of the reflection ultrasonic waves 32-A, 32-B, 32-C which occurs after a period of time has passed since the state shown in FIG. 2C.

The reflection ultrasonic wave 32-A passes through the acoustic lens 3 and is propagating through the acousto-optic propagation medium section 24 in the acousto-optic cell 2. The reflection ultrasonic wave 32-B is traveling through the acoustic lens 3. The reflection ultrasonic wave 32-C is traveling through the wave reception standoff 33.

The reflection ultrasonic wave 32 entering from the acoustic lens 3 three-dimensionally converges toward the convergence point of the acoustic lens 3 due to the lens effect of the acoustic lens 3. This is equivalent to the image formation process of acoustic image formation of the reflector 26.

Commonly, image formation of an acoustic image refers to a phenomenon that an ultrasonic wave converges due to the acoustic lens effect so that a sound wave concentrates at the convergence point. The ultrasonic wave which has converged to the convergence point then diverges. Here, a process which starts after formation of an image at the convergence point and which ends with divergence of the ultrasonic wave is defined as “image formation process”.

The reflection ultrasonic waves 32-A, 32-B shown in FIG. 2D have smaller wave packet thicknesses. This is because the acoustic lens 3 has a lower sound velocity than the subject 12. In FIG. 2C, the reflection ultrasonic waves 32-A, 32-B have a convex shape which is curved outward on the acoustic lens 3 side, while in FIG. 2D, the reflection ultrasonic waves 32-A, 32-B have a convex shape which is curved outward on the subject 12 side. This is because the waveform converges in a plane which is perpendicular to the traveling direction of the reflection ultrasonic waves 32-A, 32-B due to the lens effect of the acoustic lens 3, i.e., the convergence effect.

As will be described in detail below, the degree of divergence which occurs when the reflection ultrasonic waves 32-A, 32-B, 32-C reach the acoustic lens 3 varies depending on the distances between the vertexes A, B, C, which are the points of reflection, and the acoustic lens 3. Therefore, the point of convergence of the reflection ultrasonic waves 32-A, 32-B, 32-C by the acoustic lens 3 is not the only one, but the reflection ultrasonic waves 32-A, 32-B, 32-C converge at different points. Thus, the reflection ultrasonic wave from the inside of the subject 12 three-dimensionally converges at an acoustic image formation portion 4 to form an image. In the case where a plane sound wave is incident on the acoustic lens 3, the acoustic image formation portion 4 is present at a position that is more distant from the subject 12 than the convergence point at which the plane sound wave converges. An image mentioned herein refers to an acoustic pressure distribution in the acousto-optic propagation medium section 24 in which the shape of the reflector 26 is reflected and which has the highest acoustic pressure. Hereinafter, it is also referred to as “acoustic image”.

Further, as shown in FIG. 2D, a portion of the subject 12 from which an image derived from the reflection ultrasonic wave can be obtained is limited to a region where a wave transmission range 11 in which an ultrasonic wave transmitted from the ultrasonic wave transmitter 5 can diverge and a wave reception range 10 in which the reflection ultrasonic wave can be incident on the acoustic lens 3 overlap each other. The wave transmission range 11 is determined depending on the degree of divergence of the ultrasonic wave transmitted from the ultrasonic wave transmitter 5. The wave reception range 10 is determined depending on the characteristics of the acoustic lens 3.

<FIG. 2E>

FIG. 2E shows a state of the reflection ultrasonic waves 32-A, 32-B, 32-C which occurs after a period of time has passed since the state shown in FIG. 2D.

The reflection ultrasonic wave 32-A is in the most converged state when it passes through the acoustic image formation portion 4, and thereafter, the reflection ultrasonic wave 32-A diverges again while propagating through the acousto-optic propagation medium section 24.

The reflection ultrasonic wave 32-B is present in the acoustic image formation portion 4 and is in the most converged state. That is, the reflection ultrasonic wave 32-B exists in the form of a point corresponding to the vertex B.

The reflection ultrasonic wave 32-C has already reached the acoustic image formation portion 4 of the acousto-optic propagation medium section 24. Before this point in time, the reflection ultrasonic waves 32-A, 32-B, 32-C does not yet reach a region of the acousto-optic propagation medium section 24 which is irradiated with the convergent light 29. Therefore, the Bragg diffracted light 30 is not yet produced.

<FIG. 2F>

FIG. 2F shows a state of the reflection ultrasonic waves 32-A, 32-B, 32-C which occurs after a period of time has passed since the state shown in FIG. 2E.

The reflection ultrasonic wave 32-A reaches the inside of the convergent light 29 propagating through the acousto-optic propagation medium section 24. The reflection ultrasonic wave 32-B, which is in a divergent state, also reaches the inside of the convergent light 29 propagating through the acousto-optic propagation medium section 24. The reflection ultrasonic wave 32-C is present in the acoustic image formation portion 4 and is in the most converged state.

Since the acousto-optic propagation medium section 24 has a smaller sound velocity than the subject 12, the Bragg diffraction conditions can be sufficiently satisfied even in the case of a relatively-low ultrasonic wave frequency at which a deep inner portion of a subject which causes a large attenuation, such as an organism's body, can be imaged. Therefore, as previously described with reference to FIG. 10, Bragg diffracted light 30 is produced by each of the reflection ultrasonic waves 32-A, 32-B.

The produced Bragg diffracted light 30 is detected by the image formation optical system 14. Since the focal point of the cylindrical lens 21 is on the light receiving plane of the image sensor 22, optical images of the points A, B are formed on the light receiving plane. The image sensor 22 detects the optical images and converts them to electric signals.

The reflection ultrasonic wave 32-C thereafter diverges to reach the inside of the convergent light 29 propagating through the acousto-optic propagation medium section 24. As a result, Bragg diffracted light 30 is produced, and the image sensor 22 detects an optical image of the point C.

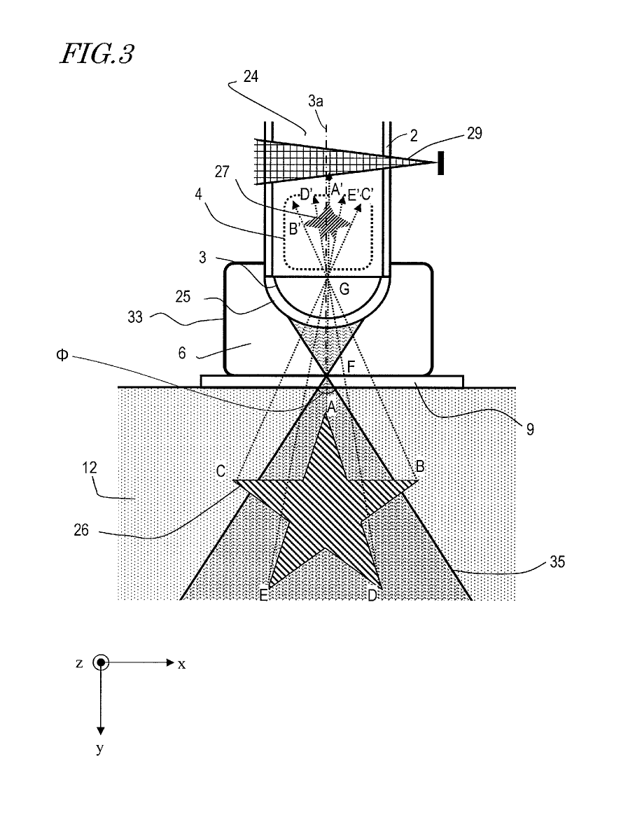

FIG. 3 shows the positional relationship between an acoustic image which is formed by the acoustic lens 3 in the acoustic image formation portion 4 of the acousto-optic propagation medium section 24 and the convergent light 29. In FIG. 3, the wave reception standoff 33 and the coupling medium 6 have sound velocities which are generally equal to the sound velocity of the subject 12. Point G shown in FIG. 3 indicates the center of curvature of the acoustic lens 3. In this embodiment 1, it has a hemispherical shape.

The range 35 shown in FIG. 3 schematically illustrates the convergence characteristics in the hemispherical shape of the acoustic lens 3. Point F shown in FIG. 3 indicates the convergence point (focal point) on the hemispherical shape side. When the point sound source is placed on the point F, a plane wave is observed in a plane which is perpendicular to the acoustic axis 3a and which includes the curvature center G of the acoustic lens 3.

In the conventional electronic scanning ultrasonic diagnostic apparatuses, ultrasonic waves transmitted from a large number of ultrasonic transducers of a probe are converged into the shape of a beam for scanning an inner portion of the subject 12. Here, an image obtained can have a higher resolution as the beam diameter decreases.

On the other hand, according to the acousto-optic imaging device 1 of the present embodiment, the acoustic lens 3 intends to form an acoustic image 27 of the reflector 26 that is present inside the subject 12 in the acoustic image formation portion 4 which is set at a position that is beyond the curvature center G, rather than improving the resolution near the point F.

To form the acoustic image in the acoustic image formation portion 4, the reflector 26 needs to be present opposite to the acoustic lens 3 relative to the point F. This is because a sound source which is present on the acoustic lens 3 side relative to the point F cannot be converged by the acoustic lens 3, such as in the case of an optical lens that cannot form a real image of an object which is present on the optical lens side relative to the focal point.

The acoustic image 27 formed in the acoustic image formation portion 4 is a three-dimensional image which is determined according to the shape of the reflector 26 and the relative position to the acoustic lens 3 (although the reflector 26 is shown as a two-dimensional image in the drawing). The acoustic lens 3 included in the acousto-optic imaging device 1 performs a totally different function from a conventional ultrasonic wave. The acoustic image 27 serves as a secondary sound source to produce a divergent ultrasonic wave again in the acousto-optic propagation medium section 24, and acousto-optic visualization of the reflector 26 is carried out based on the principle of Bragg diffraction illustrated in FIG. 10. Therefore, the position at which the acousto-optic effect occurs, i.e., the position of the convergent light 29, is beyond the acoustic image formation portion 4 relative to the acoustic lens.

The principle of Bragg diffraction holds in a xy plane at an arbitrary position on the z-axis in FIG. 3, i.e., in an arbitrary xy plane. Therefore, in an arbitrary plane which is parallel to the drawing sheet of FIG. 3, the acoustic image 27 is visualized according to the principle of Bragg diffraction.

In FIG. 3, the acoustic lens 3 has a sufficient view angle φ for visualization of the reflector 26 over a wide range. The vertexes A, B, C, D, E of the reflector 26 are present beyond the point. Therefore, acoustic images 27 are formed as points A′, B′, C′, D′, E′ in the acoustic image formation portion 4 such that the acoustic images 27 are horizontally reversed on extended lines of the line segments extending between the respective vertexes and the curvature center G. As previously described with reference to FIGS. 2A to 2F, the acoustic images 27 are not concurrently formed.

The images of the points A′, B′, C′, D′, E′ are sequentially formed in an order which follows the propagation time of the ultrasonic wave which can be calculated from the distance between the ultrasonic wave transmitter 5 and the reflector 26 and the distance between the reflector 26 and the acoustic lens 3. Therefore, the image sensor 22 also detects the acoustic images 27 (i.e., the reflector 26) in an order which accords with the order of formation of the acoustic images 27. The acoustic image 27 deforms due to the effect of the optical transfer function of the acoustic lens 3, but the deformation can be analyzed in the designing stage. Thus, after an image is obtained by the image sensor 22, it is only necessary to correct image data based on analysis results.

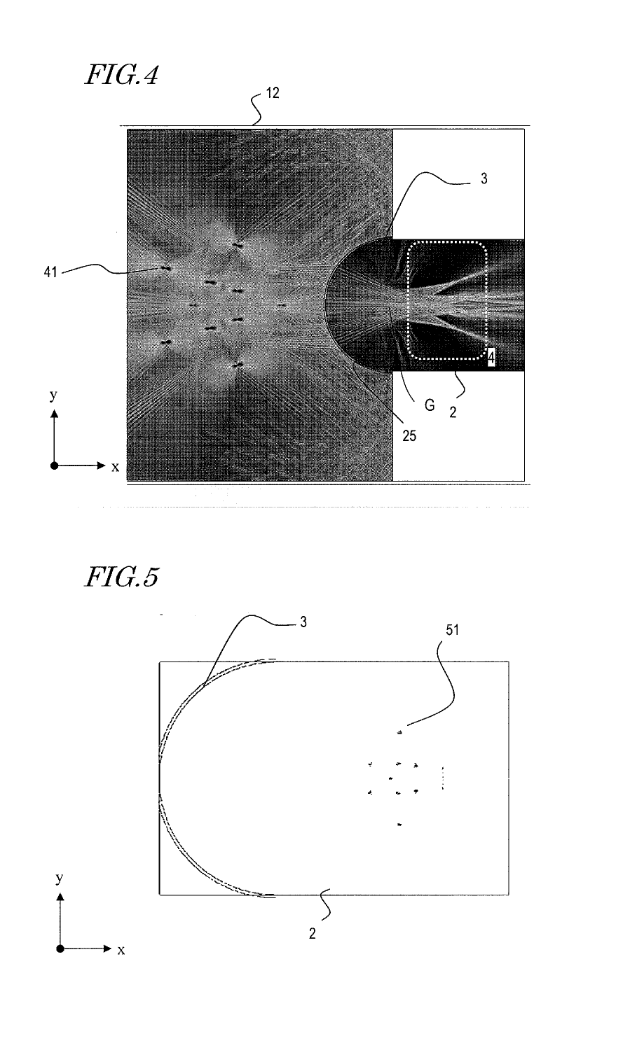

As described hereinafter, the effect of acoustic image formation which was achieved by the acoustic lens in the acousto-optic imaging device 1 of the present embodiment was confirmed by simulation. FIG. 4 shows a beam pattern of a sound wave from a point sound source which was formed by an acoustic lens. As shown in FIG. 4, ten point sound sources 41 were present at the apexes and crotches of the star shape. For the sake of convenience of simulation, on the lines extending between the respective points and the curvature center G of the acoustic lens, the point sound sources 41 had directivity in a direction toward the curvature center G and in the opposite direction thereof. Therefore, in FIG. 4, each of the point sound sources 41 seems like two sound sources.

The subject 12 was formed by water and had a density of 1 g/cc and a sound velocity of 1500 m/s. The acoustic lens 3 and the acoustic image formation portion 4 had a sound velocity of 500 m/s and a density of 1.6 g/cc. The acoustic lens 3 was covered with a thin cover layer 25 (polyethylene, sound velocity: 1950 m/s, density: 0.9 g/cc, thickness: 0.4 mm). The radius of curvature of the acoustic lens was 15 mm. The picot sound sources 41 were positioned in the range of 10 mm to 36 mm from the tip end of the acoustic lens 3. The extent in the y-direction was about 27 mm.

In FIG. 4, burst ultrasonic waves of 10 cycles which had a frequency of 5 MHz were concurrently emitted from the respective point sound sources 41, images were formed in the acoustic image formation portion 4 of the acousto-optic cell 2 via the acoustic lens 3, the length of time which elapsed after that till they diverged was calculated, and the maximum values of the acoustic pressure within a calculation period at the respective points in a calculation space are shown.

As shown in FIG. 4, in the acoustic image formation portion 4, each beam converged to form an acoustic image and thereafter diverged.

FIG. 5 shows the instantaneous acoustic pressure distribution at the time when an acoustic image was formed in the acoustic image formation portion 4. In FIG. 5, the convergence point (image formation) 51 of a sound wave is illustrated. Since the structure of the acoustic lens 3 was a simple spherical structure and sound waves were concurrently radiated from the respective sound sources, it was observed that the sound waves from the respective sound sources converged generally concurrently to form images. Note that, however, the sound source which was at the point closest to the acoustic lens did not converge but thereafter propagated as a plane wave. The conditions of the simulation were such that the focal length of the acoustic lens was about 10 mm and the point sound source was placed near the focal point, and therefore, it is inferred that image formation in the acoustic image formation portion 4 fails.

The acoustic images of the other sound sources diverge since the time shown in FIG. 5 and propagate as spherical waves in the acousto-optic cell 2. Therefore, these point sound sources can be optically imaged using a light source and an image formation optical system.

From the above researches, it was found that, in the configuration of this simulation, the minimum imaging distance is about 10 mm. Therefore, when one intends to obtain an image of an inner portion of the subject 12 immediately underneath the surface, the acoustic lens 3 may be separated by the wave reception standoff from the surface of the subject 12 by 10 mm or more.

In this simulation, the acousto-optic propagation medium section 24 of the acousto-optic cell 2 had a sound velocity of 500 m/s and a density of 1.6 g/cc. These physical properties can be realized by using, for example, Fluorinert FC-72 manufactured by 3M. Fluorinert is an inert fluid which is composed of several types of perfluorocarbons and has an extremely low reactivity with other substances, and is therefore suitably used as a constituent material of the acousto-optic propagation medium section 24 or the acoustic lens 3. When Fluorinert is used, the wavelength compression effect is about three times greater, and therefore, imaging of the subject 12 with the use of an ultrasonic wave at 5 MHz or higher, preferably at about 10 MHz, can satisfy the Bragg diffraction conditions. Thus, due to the Bragg diffraction, a reflector distribution inside the subject can be imaged.

The other example materials which can be used for the acousto-optic propagation medium section 24 include high performance materials Novec 7100 and Novec 7200 manufactured by 3M. Novec 7100 and Novec 7200 are inert fluids which are mainly composed of hydrofluoroether. Each of them has a sound velocity of about 630 m/s and a density of around 1.5 g/cc. Although the sound velocity is slightly fast and the wavelength compression effect is low as compared with Fluorinert, the conditions of Bragg diffraction can be sufficiently satisfied when using an ultrasonic wave at about 10 MHz.

The other example materials which can be used for the acousto-optic propagation medium section 24 include a nanofoam material that is a silica porous element. The nanofoam material has a density of 0.05 g/cc to 0.3 g/cc and has a sufficient light transmittance. The sound velocity of the nanofoam material is about 50 m/s to 300 m/s. Since it has an extremely low sound velocity as the solid acoustic material, it is extremely suitable as a low sound velocity material for the acousto-optic cell. Note that, however, the acoustic impedance of the silica porous element is largely different from that of the organism's body, and therefore, using an acoustic matching structure is preferred. When a nanofoam which has a sound velocity of 50 m/s is used as the acousto-optic propagation medium section 24, the wavelength of the sound wave at 10 MHz is 5 μm. When near-infrared laser light at a wavelength of 1.5 μm is used as the light source, the Bragg diffraction angle is about 8°. In Non-patent Document 1, the Bragg angle is about 0.3°, and the separation distance from the zeroth order light can be greatly reduced by increasing the diffraction angle. Since large part of the dimensions of the image formation optical system is the distance arranged for separation of zeroth order light and diffracted light, introduction of a nanofoam acousto-optic cell enables to greatly reduce the size of the image formation optical system.

The acoustic lens 3, the acousto-optic propagation medium section 24, and the acoustic image formation portion 4 may be made of the same material or may be made of different materials. The respective sections may be made of different materials so long as the acoustic pressure and the wavelength compression effect are secured such that Bragg diffraction occurs in the acousto-optic propagation medium section 24. When the acoustic lens 3 is made of a fluid material such as Fluorinert or Novec, the surface of the acoustic lens 3 is preferably provided with a cover layer 25. A preferred material for the cover layer is a plastic material, such as polyethylene, polystyrene, or the like.

Although the acoustic lens 3 has a hemispherical shape, it may have a shape of a dome which is smaller than a hemisphere or an aspherical shape and may be a solid lens which is made of a resin material or composite material so long as it is such an acoustic lens that a predetermined wave reception range is secured and an acoustic image is formed. As the coupling medium 8 in the wave transmission standoff 7 and the coupling medium 6 in the wave reception standoff 33, degassed water or various oils may be used. For the window 9, polystyrene or industrial plastic, such as PET, PPS, etc., may be preferably used from the viewpoint of the acoustic matching property between the organism's body and the coupling medium.

According to the acousto-optic imaging device of the present embodiment, a divergent ultrasonic wave is transmitted toward an inner portion of a subject, a reflection ultrasonic wave obtained from the inner portion is converged by an acoustic lens, and the wave which is in a divergent state after the convergence is caused to propagate through an acousto-optic propagation medium section. The reflection ultrasonic wave propagating through the acousto-optic propagation medium section, which is in a state of a divergent wave, is irradiated with convergent light, whereby diffracted light which is attributed to Bragg diffraction can be obtained. Therefore, an image of the inner portion of the subject can be optically obtained at a high speed without carrying out complicated signal processing on the ultrasonic wave.

Since the sound velocity of the acousto-optic propagation medium section is smaller than the sound velocity of the subject, the wavelength of the ultrasonic wave propagating through the acousto-optic propagation medium section is shorter than that of the ultrasonic wave propagating through the subject. Thus, the frequency of the ultrasonic wave transmitted from the ultrasonic wave transmitter can be decreased, and a low frequency ultrasonic wave that is unlikely to attenuate inside the subject can be used.

### Second Embodiment

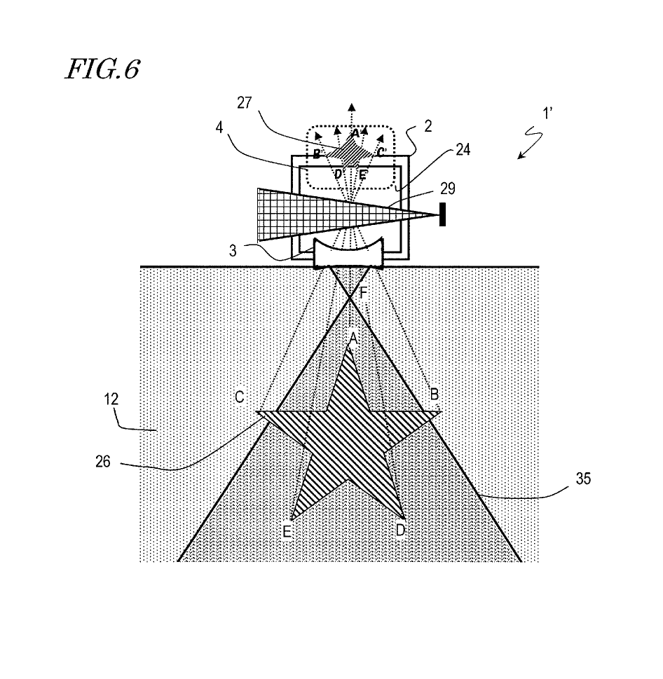

FIG. 6 is a schematic diagram showing major part of the second embodiment of the acousto-optic imaging device of the present invention. In the acousto-optic imaging device 1′ of the present embodiment, the position where the convergent light 29 and a reflection wave propagating through the acousto-optic propagation medium section 24 interact is different from that of the first embodiment. The ultrasonic wave transmitter 5, the light source 13, and the image formation optical system 14 have the same configurations as those of the first embodiment and are therefore not shown in FIG. 6.

As shown in FIG. 6, the acousto-optic imaging device 1′ includes a biconcave acoustic lens 3 which is made of a resin. The convergent light 29 is propagating through the acousto-optic propagation medium section 24 between the acoustic image formation portion 4 and the acoustic lens 3. In the present embodiment, the convergent light 29 passes through a region through which the reflection ultrasonic wave in a state of a convergent wave is propagating, thereby producing Bragg diffracted light.

In FIG. 10, when the propagation direction of the ultrasonic wave radiated from the point O₁ is reversed, it will be an acoustic wave which converges at the point O₁, and the point O₁ can be assumed as a convergence point of a converged sound wave. Therefore, in the interaction region of the ultrasonic wave and the light (from the point S₁ to the point S₃), there is no geometrical change except that the propagation direction of the ultrasonic wave is reverse and the ultrasonic wave is in a state of a convergent wave, and Bragg diffraction of the light which is attributed to the ultrasonic wave occurs likewise so that a diffraction image is formed at the point O₃. Note that, however, the diffracted light produced herein is the +1st order light, and the diffraction image is a +1st order diffraction image, although there is no substantial difference between the +1st order diffraction image and the −1st order diffraction image. Thus, in the configuration shown in FIG. 6, an inner portion of the subject 12 can also be imaged as in the first embodiment.

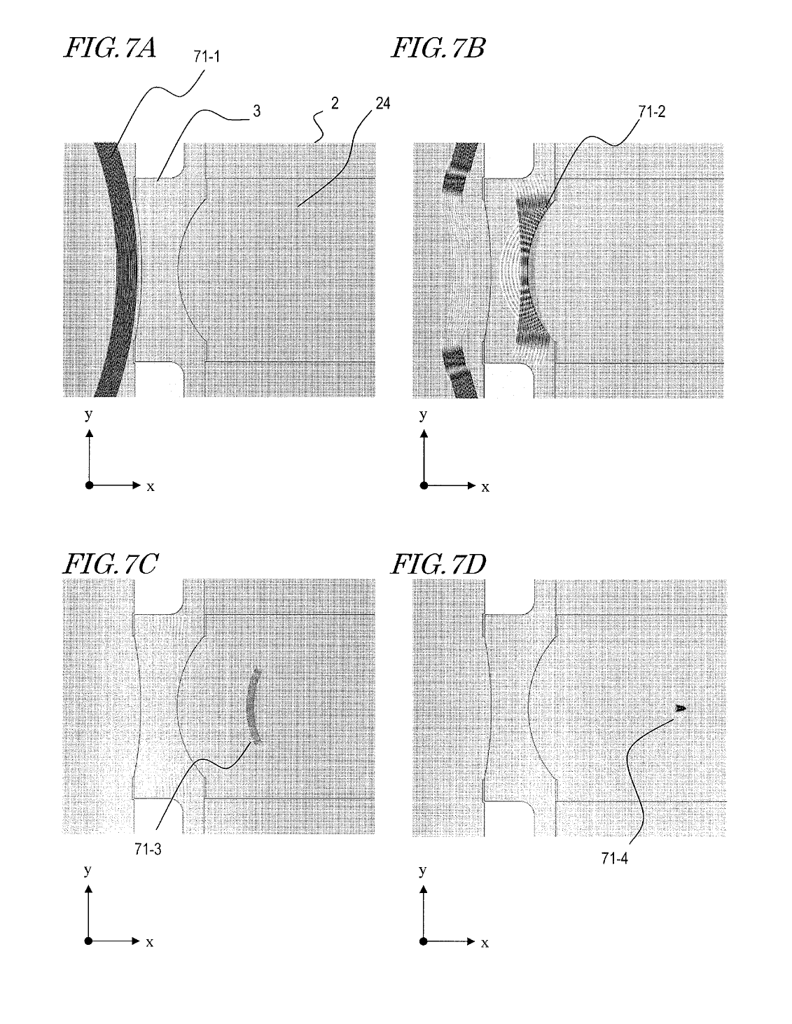

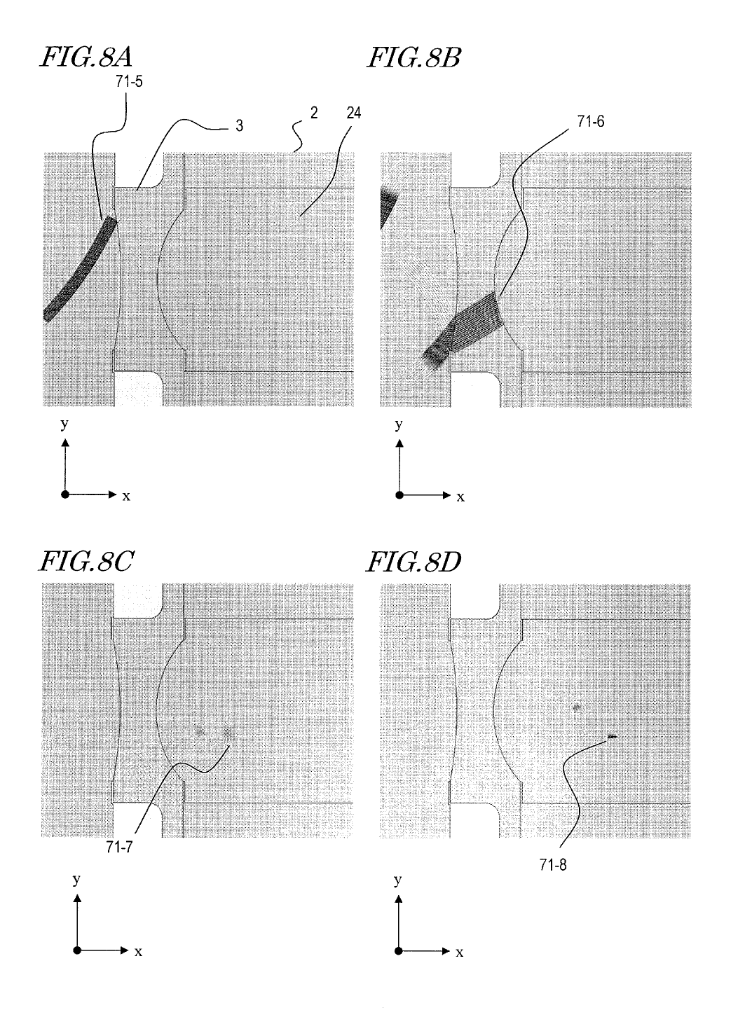

FIG. 7 and FIG. 8 show the variation over time of the acoustic pressure distribution by the acoustic lens of the second embodiment, which was evaluated by simulation. The acoustic lens 3 was a biconcave acoustic lens. The width of the concave portion (lens aperture width) was 20 mm on both sides. The radius of curvature on the subject 12 side was 52 mm. The radius of curvature on the acousto-optic cell side was 14.8 mm. The thickness of the lens was 10 mm. The lens was made of polystyrene (density: 1.05 g/cc, longitudinal wave sound velocity: 2400 m/s, transverse wave sound velocity 1050 m/s). The acousto-optic propagation medium section 24 of the acousto-optic cell 2 was made of high performance fluid Novec 7200 (density: 1.43 g/cc, sound velocity: 623 m/s) manufactured by 3M. The width of the acousto-optic propagation medium section 24 (y-direction of the drawing) was 26 mm. The dimension of the ultrasonic wave along the propagation direction (x-direction of the drawing) was 24 mm. The focal length in Novec 7200 was 15 mm. The medium on the subject side was water (density: 1 g/cc, sound velocity: 1496 m/s).

FIGS. 7A to 7D show image formation of an ultrasonic wave 71 in the case where it was placed at a point sound source (point reflector) at the distance of 60 mm from the acoustic lens with an angle of 0 degree. A burst transmission wave of 10 cycles was used at a frequency of 5 MHz. FIG. 7A shows the acoustic pressure distribution at the time immediately before the ultrasonic wave 71-1 from the sound source entered the acoustic lens. The ultrasonic wave 71-1 was diverging convexly in the propagation direction. FIG. 7B shows a state that the ultrasonic wave 71-2 that was incident on the acoustic lens 3 partially penetrated into the acousto-optic propagation medium section 24. Since the acoustic lens 3 had a greater sound velocity than water (subject), the wave packet of the ultrasonic wave 71-2 in the lens was elongated in the propagation direction. FIG. 7C shows a state that the entire wave packet of the ultrasonic wave 71-3 penetrated into the acousto-optic propagation medium section 24 and was propagating therethrough. Due to the wavelength compression effect of the acousto-optic propagation medium section 24, the wave packet was compressed in the propagation direction. Due to the lens effect of the acoustic lens 3, it was concave relative to the propagation direction, and the ultrasonic wave was in a converged state. FIG. 7D shows a state that the ultrasonic wave 7-4 completely converged to form an acoustic image (point sound source). The distance from the acoustic lens was about 18 mm. Therefore, the convergent light 29 may be placed near the ultrasonic wave 71-3 shown in FIG. 7C.

FIG. 8 shows the result of a calculation with a point sound source placed at a distance of 60 mm with an angle of +30 degrees (the direction to the upper left corner of the drawing), with the other conditions being the same as those of the simulation illustrated in FIG. 7. FIG. 8A shows a state immediately before the ultrasonic wave 71-5 entered the acoustic lens 3. Although the propagation direction of the ultrasonic wave 71-5 was inclined according to the position of the sound source, the ultrasonic wave 71-5 was diverging convexly relative to the propagation direction. Here, for the sake of convenience of calculation, part of the sound wave in part of the drawing lying above the aperture of the acoustic lens 3 was neglected. FIG. 8B shows a state that large part of the ultrasonic wave 71-6 entered the acoustic lens 3, and the ultrasonic wave partially penetrated into the acousto-optic propagation medium section 24. Since the ultrasonic wave was diagonally incident, the acoustic pressure decreased. FIG. 8C shows a state that the ultrasonic wave 71-7 entirely penetrated into the acousto-optic propagation medium section 24 and was propagating through the acousto-optic propagation medium section 24. The wavefront was convex relative to the propagation direction. An ultrasonic wave observed other than the ultrasonic wave 71-7 which was in a converged state was a wave packet. This is a wave packet which occurs as a result of multiple reflection in the acoustic lens 3 and constitutes a cause of artifacts. FIG. 8D shows a state that the ultrasonic wave 71-8 sufficiently converged to form an acoustic image. The distance from the acoustic lens 3 was about 13 mm. Under the conditions of the simulation illustrated in FIG. 8, an inner portion of the subject in an azimuth of about ±30 degrees can be visualized to a depth of about 60 mm when the convergent light 29 is placed at a distance of around 10 mm from the acoustic lens.

According to the present embodiment, convergent light is allowed to pass through a portion of the acousto-optic propagation medium section 24 between the acoustic image formation portion 4 and the acoustic lens 3. Therefore, the size of the acousto-optic cell 2 can be decreased as compared with the first embodiment. Since the acoustic image formation portion 4 is not utilized, it is not necessary to provide the acoustic image formation portion 4 in the acousto-optic cell 2. When the size reduction of the acousto-optic cell 2 leads to a problem of multiple reflection of an ultrasonic wave, for example, a sound absorbing structure, such as a sound absorbing element or wedges, may be provided at an appropriate position in the acousto-optic cell 2 such that unnecessary waves caused by multiple reflection or the like are reduced. Further, as in the first embodiment, a standoff may be provided so as to separate the acoustic lens from the subject. With this arrangement, an image of a shallower portion of the subject can be formed.

The acousto-optic propagation medium section 24 provided in the acousto-optic cell 2 generally has a large sound wave attenuation characteristic as compared with water. For example, Fluorinert FC-72 exhibits an attenuation characteristic of about 0.5 dB/mm at 10 MHz, and Novec 7200 exhibits an attenuation characteristic of about 0.2 dB/mm at 10 MHz. The nanofoam material, which is a solid material, exhibits an attenuation characteristic of 1 dB to 3 dB/mm. Therefore, for example, when the reflection level is small due to the attenuation characteristic of the subject, or when a large wave transmission level cannot be secured due to subject-related factors, attenuation of the reflection ultrasonic wave in the acousto-optic propagation medium section 24 can be a problem. According to the present embodiment, the distance that the reflection ultrasonic wave propagates through the acousto-optic propagation medium section 24 can be shortened, and therefore, the effect of attenuation is reduced, and a wide range image of an inner portion of the subject can be obtained under desirable conditions.

An acousto-optic imaging device disclosed in the present application is suitably used for a medical ultrasonic diagnostic apparatus. Particularly, still faster imaging than conventional ultrasonic diagnostic apparatuses is possible, and it is particularly useful in the fields of functional diagnosis for dynamic organs, such as heart. Further, it is also useful as a nondestructive inspection apparatus.

While the present invention has been described with respect to preferred embodiments thereof, it will be apparent to those skilled in the art that the disclosed invention may be modified in numerous ways and may assume many embodiments other than those specifically described above. Accordingly, it is intended by the appended claims to cover all modifications of the invention that fall within the true spirit and scope of the invention.

What is claimed is:

1. An acousto-optic imaging device, comprising:
an ultrasonic wave transmitter for transmitting a divergent ultrasonic wave into a subject;
an acoustic lens for converging a reflection ultrasonic wave derived from the ultrasonic wave from the subject;
an acousto-optic cell including an acousto-optic propagation medium section which has a smaller sound velocity than the subject and through which the reflection ultrasonic wave converged by the acoustic lens propagates;
a light source for emitting convergent light so as to irradiate the reflection ultrasonic wave propagating through the acousto-optic propagation medium section in a direction not parallel to a traveling direction of the reflection ultrasonic wave; and
an image formation optical system for detecting Bragg diffracted light of the convergent light which is produced in the acousto-optic propagation medium section and converting the detected Bragg diffracted light to an electric signal.

2. The acousto-optic imaging device of claim 1, wherein the convergent light is emitted so as to irradiate a portion of the acousto-optic propagation medium section through which the reflection ultrasonic wave in a state of a divergent wave after convergence is propagating.

3. The acousto-optic imaging device of claim 1, wherein the convergent light is emitted so as to irradiate a portion of the acousto-optic propagation medium section through which the reflection ultrasonic wave in a state of a convergent wave is propagating.

4. The acousto-optic imaging device of claim 1, wherein the acousto-optic propagation medium section includes an inert perfluorocarbon fluid.

5. The acousto-optic imaging device of claim 1, wherein the acousto-optic propagation medium section includes an inert hydrofluoroether fluid.

6. The acousto-optic imaging device of claim 1, wherein the acousto-optic propagation medium section includes a silica nanoporous element.

7. The acousto-optic imaging device of claim 1, further comprising a wave reception standoff for supporting the acoustic lens,
wherein a convergence point on a subject side of the acoustic lens occurs inside the wave reception standoff.

8. The acousto-optic imaging device of claim 1, further comprising a wave transmission standoff for supporting the ultrasonic wave transmitter,
wherein the ultrasonic wave transmitter transmits a converging ultrasonic wave, and a point of the convergence occurs inside the wave transmission standoff.
